(19) United States
(12) Patent Application Publication
(10) Pub. No.: US 2023/0363746 A1
(43) Pub. Date: Nov. 16, 2023

(54) NOVEL NON-CLOGGING SWEAT SENSING DEVICE AND METHODS OF MAKING THE SAME

(71) Applicant: THE COCA-COLA COMPANY, Atlanta, GA (US)

(72) Inventors: Steven Charles DEANE, Harston, Cambridgeshire (GB); Thore Mainart BÜCKING, Harston, Cambridgeshire (GB); Samuel Luke JOHNSTONE, Harston, Cambridgeshire (GB)

(21) Appl. No.: 18/030,882

(22) PCT Filed: Oct. 18, 2021
(86) PCT No.: PCT/US2021/055417

§ 371 Date: Apr. 7, 2023

Related U.S. Application Data
(60) Provisional application No. 63/093,435, filed on Oct. 19, 2020.

(51) Int. Cl. A61B 10/00; A61B 5/145; A61B 5/0537; A61B 5/00

(52) U.S. Cl. CPC A61B 10/0064; A61B 5/14517; A61B 5/0537; A61B 5/742; A61B 2562/125

(57) ABSTRACT

Disclosed herein are devices for measuring sweat that are substantially clogging free, having low dead volumes, and allowing a quick sensory response. Also disclosed herein are methods of making and using such devices.

### CROSS-REFERENCE TO RELATED APPLICATIONS

This application claims benefit of U.S. Provisional Application No. 63/093,435, filed Oct. 19, 2020, the contents of which is incorporated herein by reference in its entirety.

### TECHNICAL FIELD

The present invention generally relates to devices configured to measure and monitor sweat and methods of making the same. The present disclosure also relates to methods of measuring sweat using the disclosed herein sweat sensing devices.

### BACKGROUND

It is often advantageous to monitor and carry out an analysis of both the rate of production and the composition of a person's sweat while they are performing strenuous physical exertion since such knowledge enables the individual to more accurately replenish lost sweat fluid and electrolytes.

However, sweat varies in composition between individuals, and for a given individual, the sweat composition can depend on a sweating rate, which is itself a function of temperature, humidity, exertion rate, etc. Sports fluid replacement drinks help maintain electrolyte balance, but the optimum level of electrolytes for a given individual in their current state is variable. Too high or too low electrolyte levels can hurt taste perception, as well as negatively affect the body, and the individual needs to be educated to select the optimum beverage to maintain their physiology in the optimum state.

Thus, it is important to enable an individual to determine their electrolyte loss by sweating and so help them replenish their electrolytes optimally, enabling further exertion to be maximized. This knowledge would help support better competitive performance and also more effective training regimes.

However, there is a challenge to precisely analyze sweat due to the low sweat rate. It is understood that the devices used to analyze sweat need to measure very small sample volumes. A consequence of the small volume requirement is that the device is likely to get blocked (clogged) during repeated use as sweat contains dissolved salts and other non-dissolvable constituents. The simplest solution to this is bringing the exposed sensor into intimate contact with the skin, allowing for fast sweat refreshing rates to minimize the blockage. The problem with this is a possible interference from the subject's skin, and therefore, the device needs to be specific to the sweat rather than a combination of sweat and skin.

Accordingly, a need exists for devices capable of accurately measuring sweat in small volumes without interference from the skin. There is also a need for devices that are reusable, free of clogging, and simple for use and manufacture. These needs and other needs are at least partially satisfied by the present disclosure.

### SUMMARY

The present invention is directed to a device comprising: a) a sweat collector having a shape, wherein at least a portion of the sweat collector comprises a first portion configured to face and to conform to a subject's skin; b) a sealing member configured to encompass the sweat collector and form a seal between the sweat collector and the subject's skin; c) an open channel having a width, and a height, wherein the open channel has an aspect ratio of the height to the width no greater than about 10; wherein the channel is configured to continuously receive and transfer through an aliquot of a collected sweat from the sweat collector; and d) a sensor that is in fluid communication with the collected sweat and is configured to detect at least one property of the collected sweat, wherein the sensor is positioned at a distance from the first portion such that the sensor has substantially no contact with the subject's skin.

In still further aspects, the sensor of the disclosed device is configured to detect at least one property of the sweat selected from impedance, conductivity, refraction, temperature, or a combination thereof. In such exemplary aspects disclosed herein, the sensor can be an impedance sensor.

While in other aspects, the impedance sensor, as disclosed herein, can comprise two electrodes, each having an electrode length and positions opposing each other at an electrode gap.

Also disclosed herein is a device comprising: a) a sweat collector having a shape, wherein at least a portion of the sweat collector comprises a first portion configured to face and to conform to a subject's skin; b) a sealing member configured to encompass the sweat collector and form a seal between the sweat collector and the subject's skin; c) an open channel having a width, and a height, wherein the channel has an aspect ratio of the height to the width no greater than about 10; wherein the channel is configured to continuously receive and transfer through an aliquot of a collected sweat from the sweat collector; and d) a sensor comprising two electrodes, each having an electrode length and positioned opposing each other at an electrode gap and are in fluid communication with the collected sweat and wherein the sensor is configured to detect an impedance of the collected sweat and wherein the sensor is positioned at a distance from the first portion such that the sensor has substantially no contact with the subject's skin.

Still further disclosed herein is a method of measuring a sweat comprising: providing any of the disclosed herein devices, wherein the device is positioned on a subject's skin such that the sealing member seals the sweat collector against the subject's skin; collecting the sweat within a sweat collection area; and measuring at least one property of the sweat by the sensor, wherein the at sensor provides one or more output signals correlated with the at least one property of the sweat.

Also disclosed herein are methods of making the disclosed devices. In certain aspects, disclosed herein is a method comprising: a) forming a sweat collector on a rigid material; b) positioning a sealing member such that the sealing member is configured to encompass the sweat collector; c) forming an open channel, wherein the open channel has a width and a height, wherein the open channel has an aspect ratio of the height to the width no greater than about 10; wherein the open channel is configured to continuously receive and transfer through an aliquot of a collected sweat from the sweat collector; and d) positioning a sensor such that the sensor is positioned at a distance from the first portion such that the sensor has substantially no contact with the subject's skin and such that the sensor is in fluid communication with the collected sweat and is configured to detect at least one property of the collected sweat.

Additional aspects of the disclosure will be set forth, in part, in the detailed description, figures, and claims which follow, and in part will be derived from the detailed description, or can be learned by practice of the invention. It is understood that both the foregoing general description and the following detailed description are exemplary and explanatory only and are not restrictive of the invention as disclosed.

### BRIEF DESCRIPTION OF DRAWINGS

FIG. 1 depicts a schematic of an exemplary device applied to the skin in one aspect.

FIG. 2A depicts a top view of an exemplary device in one aspect.

FIG. 2B depicts a side view of an exemplary device in one aspect.

FIG. 3A-3D depicts a schematic of an exemplary device in one aspect.

FIG. 4 depicts a photograph of a sensor on an exemplary device in one aspect.

FIG. 5 depicts the effect of skin interference on a device response for different electrode spacing and positioning from the skin.

FIG. 6 shows a plot of a measured impedance at a range of physiologically realistic sweat concentrations.

FIG. 7 depicts exemplary impedance measurements of saline at different concentrations representative of sweat. Measurements were made at both 10 kHz and 100 kHz (small and large circles, respectively).

FIG. 8 depicts the detection of unclean sensor state in one aspect.

### DETAILED DESCRIPTION

The present invention can be understood more readily by reference to the following detailed description, examples, drawings, and claims, and their previous and following description. However, before the present articles, systems, and/or methods are disclosed and described, it is to be understood that this invention is not limited to the specific or exemplary aspects of articles, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known aspect. To this end, those skilled in the relevant art will recognize and appreciate that many changes can be made to the various aspects of the invention described herein while still obtaining the beneficial results of the present invention. It will also be apparent that some of the desired benefits of the present invention can be obtained by selecting some of the features of the present invention without utilizing other features. Accordingly, those of ordinary skill in the pertinent art will recognize that many modifications and adaptations to the present invention are possible and may even be desirable in certain circumstances and are a part of the present invention. Thus, the following description is again provided as illustrative of the principles of the present invention and not in limitation thereof.

### Definitions

As used herein, the singular forms “a,” “an,” and “the” include plural referents unless the context clearly dictates otherwise. Thus, for example, reference to a “device” or an “electrode” includes aspects having two or more such devices or electrodes unless the context clearly indicates otherwise.

It is appreciated that certain features of the disclosure, which are, for clarity, described in the context of separate aspects, can also be provided in combination in a single aspect. Conversely, various features of the disclosure, which are, for brevity, described in the context of a single aspect, can also be provided separately or in any suitable subcombination.

As used herein, the terms “optional” or “optionally” mean that the subsequently described event or circumstance may or may not occur, and that the description includes instances where said event or circumstance occurs and instances where it does not.

It is also to be understood that the terminology used herein is for the purpose of describing particular aspects only and is not intended to be limiting. As used in the specification and in the claims, the term “comprising” can include the aspects “consisting of” and “consisting essentially of.” Unless defined otherwise, all technical and scientific terms used herein have the same meaning as commonly understood by one of ordinary skill in the art to which this invention belongs. In this specification and in the claims, which follow, reference will be made to a number of terms that shall be defined herein.

For the terms “for example” and “such as,” and grammatical equivalences thereof, the phrase “and without limitation” is understood to follow unless explicitly stated otherwise.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value and/or to “about” another particular value. When such a range is expressed, another aspect includes from the one particular value and/or to the other particular value.

Similarly, when values are expressed as approximations, by use of the antecedent “about,” it will be understood that the particular value forms another aspect. It will be further understood that the endpoints of each of the ranges are significant both in relation to the other endpoint and independently of the other endpoint. Unless stated otherwise, the term “about” means within 5% (e.g., within 2% or 1%) of the particular value modified by the term “about.”

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6 and any whole and partial increments therebetween. This applies regardless of the breadth of the range.

It will be understood that when an element is referred to as being “connected” or “coupled” to another element, it can be directly connected or coupled to the other element, or intervening elements may be present. In contrast, when an element is referred to as being “directly connected” or “directly coupled” to another element, there are no intervening elements present. Other words used to describe the relationship between elements or layers should be interpreted in a like fashion (e.g., “between” versus “directly between,” “adjacent” versus “directly adjacent,” “on” versus “directly on”). As used herein, the term “and/or” includes any and all combinations of one or more of the associated listed items.

It will be understood that, although the terms “first,” “second,” etc., may be used herein to describe various elements, components, regions, layers, and/or sections. These elements, components, regions, layers, and/or sections should not be limited by these terms. These terms are only used to distinguish one element, component, region, layer, or section from another element, component, region, layer, or a section. Thus, a first element, component, region, layer, or section discussed below could be termed a second element, component, region, layer, or section without departing from the teachings of example embodiments.

Spatially relative terms, such as “beneath,” “below,” “lower,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” can encompass both an orientation of above and below. The device may be otherwise oriented (rotated 90 degrees or at other orientations), and the spatially relative descriptors used herein interpreted accordingly.

As used herein, the term or phrase “effective,” “effective amount,” or “conditions effective to” refers to such amount or condition that is capable of performing the function or property for which an effective amount or condition is expressed. As will be pointed out below, the exact amount or particular condition required will vary from one embodiment to another, depending on recognized variables such as the materials employed and the processing conditions observed. Thus, it is not always possible to specify an exact “effective amount” or “condition effective to.” However, it should be understood that an appropriate effective amount will be readily determined by one of ordinary skill in the art using only routine experimentation.

As used herein, the term “substantially” means that the subsequently described event or circumstance completely occurs or that the subsequently described event or circumstance generally, typically, or approximately occurs.

Still further, the term “substantially” can in some aspects refer to at least about 80%, at least about 85%, at least about 90%, at least about 91%, at least about 92%, at least about 93%, at least about 94%, at least about 95%, at least about 96%, at least about 97%, at least about 98%, at least about 99%, or about 100% of the stated property, characteristic, component, composition, or other condition for which substantially is used to characterize or otherwise quantify an amount.

In other aspects, as used herein, the term “substantially free,” when used in the context of clogging of a channel, for example, is intended to refer to a channel that is less than about 5% clogged, less than about 4% clogged, less than about 3% of clogged, less than about 2% clogged, less than about 1% clogged, less than about 0.5 clogged, less than about 0.1% clogged, or less than about 0.01% clogged.

In other aspects, as used herein, the term “substantially no,” when used in the content of contact of a disclosed surface with any other surface, for example, is intended to refer to that the disclosed surface does not contact any other surface or it contacts in a manner that does affect any properties of either surface or device.

As used herein, the term “substantially,” in, for example, the context “substantially identical” or “substantially similar” refers to a method or a system, or a component that is at least about 90%, at least about 91%, at least about 92%, at least about 93%, at least about 94%, at least about 95%, at least about 96%, at least about 97%, at least about 98%, at least about 99%, or about 100% by similar to the method, system, or the component it is compared to.

As used herein, the term “sweat” refers to a biofluid that is primarily sweat, such as eccrine or apocrine sweat, and may also include mixtures of biofluids such as sweat and blood, or sweat and interstitial fluid, or sweat and any other fluid that can be found in its vicinity, so long as advective transport of the biofluid mixtures (e.g., flow) is primarily driven by sweat.

As used herein, the term “measured” can refer in some aspects an exact or precise quantitative measurement, while in other aspects, it can also refer to measuring relative amounts, rates of change, or qualitative data. It is understood that any value that is measured can be presented in any form. In certain aspects, the data can be presented as a final concentration, as a range, as a qualitative response of “yes” or “no,” or any other form that conveys any sought information.

Numerous other general purpose or special purpose computing devices environments or configurations can be used. Examples of well-known computing devices, environments, and/or configurations that can be suitable for use include, but are not limited to, personal computers, server computers, handheld or laptop devices, smartphones, multiprocessor systems, microprocessor-based systems, network personal computers (PCs), minicomputers, mainframe computers, embedded systems, distributed computing environments that include any of the above systems or devices, and the like.

Computing devices, as disclosed herein, can contain communication connection(s) that allow the device to communicate with other devices. Computing devices can also have input device(s) such as a keyboard, mouse, pen, voice input device, touch input device, etc. Output device(s) such as a display, speakers, printer, etc., can also be included. All these devices are well known in the art and need not be discussed at length here.

Computer-executable instructions, such as program modules being executed by a computer, can be used. Generally, program modules include routines, programs, objects, components, data structures, etc., that perform particular tasks or implement particular abstract data types. Distributed computing environments can be used where tasks are performed by remote processing devices that are linked through a communications network or other data transmission medium. In a distributed computing environment, program modules and other data can be located in both local and remote computer storage media, including memory storage devices.

In its most basic configuration, a computing device typically includes at least one processing unit and memory. Depending on the exact configuration and type of computing device, memory can be volatile (such as random-access memory (RAM)), non-volatile (such as read-only memory (ROM), flash memory, etc.), or some combination of the two.

Computing devices can have additional features/functionality. For example, a computing device can include additional storage (removable and/or non-removable), including, but not limited to, magnetic or optical disks or tape.

Computing device typically includes a variety of computer-readable media. Computer-readable media can be any available media that can be accessed by the device and includes both volatile and non-volatile media, removable and non-removable media.

Computer storage media include volatile and non-volatile, and removable and non-removable media implemented in any method or technology for storage of information such as computer-readable instructions, data structures, program modules, or other data. Memory, removable storage, and non-removable storage are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computing device. Any such computer storage media can be part of a computing device.

Computing devices, as disclosed herein, can contain communication connection(s) that allow the device to communicate with other devices. The connection can be wireless or wired. Computing devices can also have input device(s) such as a keyboard, mouse, pen, voice input device, touch input device, etc. Output device(s) such as a display, speakers, printer, etc., can also be included. All these devices are well known in the art and need not be discussed at length here.

It should be understood that the various techniques described herein can be implemented in connection with hardware components or software components or, where appropriate, with a combination of both. Illustrative types of hardware components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. The methods and apparatus of the presently disclosed subject matter, or certain aspects or portions thereof, can take the form of program code (i.e., instructions) embodied in tangible media, such as CD-ROMs, hard drives, or any other machine-readable storage medium where, when the program code is loaded into and executed by a machine, such as a computer, the machine becomes an apparatus for practicing the presently disclosed subject matter.

While aspects of the present invention can be described and claimed in a particular statutory class, such as the system statutory class, this is for convenience only and one of ordinary skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred in any respect. This holds for any possible non-express basis for interpretation, including matters of logic with respect to arrangement of steps or operational flow, plain meaning derived from grammatical organization or punctuation, or the number or type of aspects described in the specification.

Moreover, for the sake of simplicity, the attached figures cannot show the various ways (readily discernable, based on this disclosure, by one of ordinary skill in the art) in which the disclosed system, method, and apparatus can be used in combination with other systems, methods, and apparatuses. Additionally, the description sometimes uses terms such as “produce” and “provide” to describe the disclosed method. These terms are high-level abstractions of the actual operations that can be performed. The actual operations that correspond to these terms can vary depending on the particular implementation and are, based on this disclosure, readily discernible by one of ordinary skill in the art.

The present invention may be understood more readily by reference to the following detailed description of various aspects of the invention and the examples included therein and to the Figures and their previous and following description.

### Device

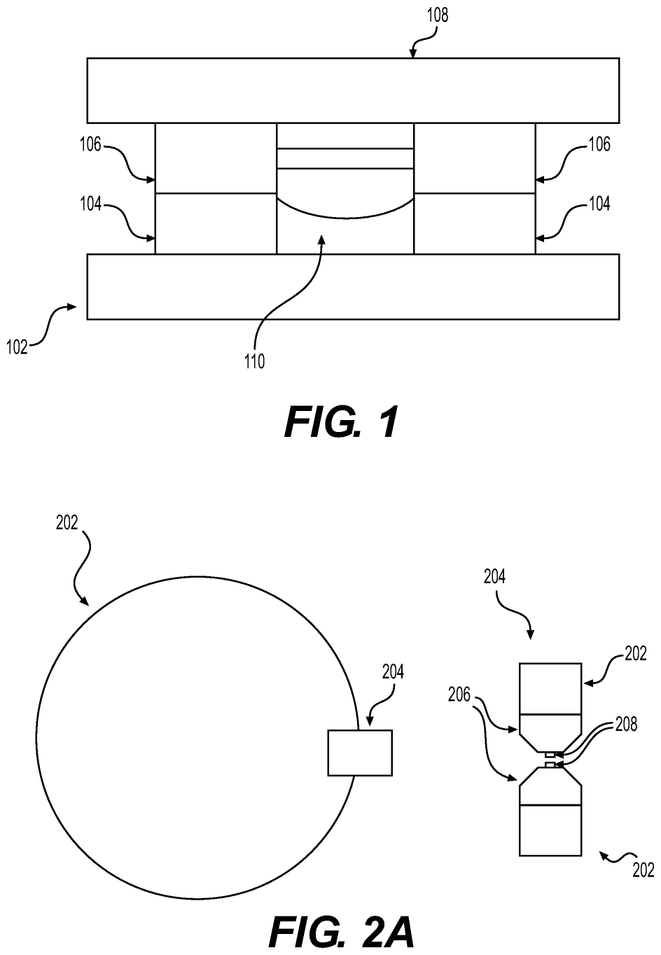

In some aspects, disclosed herein is a device configured to collect sweat from a surface on a subject's skin and guide it to a sensing area disposed at a fixed distance from the skin. A general schematic of the sensing area of an exemplary and unlimiting device is shown in FIG. 1. It can be seen that the device is sealed against the subject's skin 102 with a sealing member 104, forming an area where the sweat can be collected. A sensor 106 that is in communication with the devices' substrate 108 is positioned at a distance from the subject's skin such that there is substantially no contact between the sensor and the subject's skin, thereby minimizing skin interference in the sweat analysis. The device is built to ensure that the electric field 110 formed between the two electrodes does not reach the skin, and therefore no interference from the skin is detected.

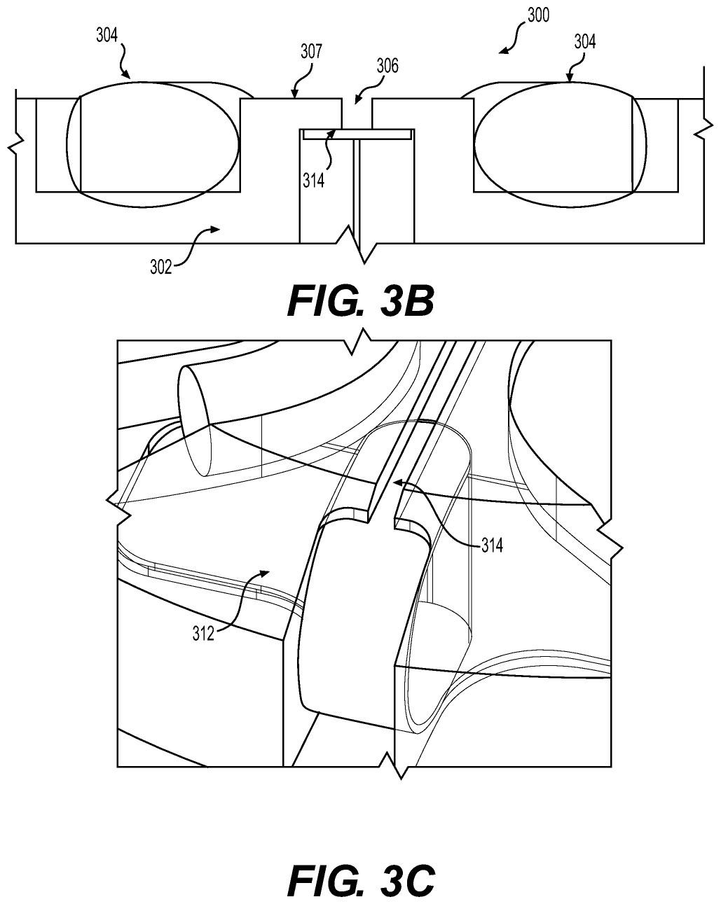

The devices of the current disclosure are further described in more detail. For example, some of the disclosed devices are shown in FIGS. 3A-3D. In certain aspects, the disclosed herein is a device 300 having a rigid support material 302. In still further aspects, the device 300 comprises a sweat collector 308. This sweat collector can be a part of the rigid support material, or it can be formed using any other materials that are separate or different from the rigid support. In yet further aspects, the sweat collector comprises a rigid material that can be the same or different from the rigid support materials. In yet other aspects, the rigid material can comprise any rigid polymer suitable for such an application. In still other aspects, the rigid material can comprise metal, ceramic, ceramic glass, wood, stone, or composite materials. It is understood that any material that allows easy manufacturing and can maintain the desired shape during manufacturing and use can be utilized. In still other aspects, it is understood that any materials that can be comfortable for the user's skin and substantially insoluble in any fluids such as sweat, blood, oils, sunscreens, lotions, and the like. In still further aspects, any chemically inert rigid materials can be utilized. In still other exemplary and unlimiting aspects, any materials that can be formed by injection molding can be utilized.

In still further aspects, the sweat collector can have a shape. In yet other aspects, the sweat collector can have a predetermined shape. It is understood, however, a specific (predetermined or not) shape of the sweat collector can change depending on the operational conditions. The exact shape of the sweat collector can be chosen based on the specific application, need to integrate the sweat collector with other potential devices, or manufacturing and design convenience. In certain aspects, the shape of the sweat collector can be substantially circular, rectangular, triangular, trapezoid, or any other irregular shape. It is understood that at least a portion of the sweat collector 308 comprises a first portion 307 that is configured to face a subject's skin (not shown). It is further understood that in certain aspects, the sweat collector can have substantially no contact with the subject's skin, or it can have a minimal contact with the subject's skin. In still further aspects, where the sweat collector can substantially contact the subject's skin. In yet other aspects, it is understood that if the sweat collector has contact with the subject's skin, such contact does not affect the device's capabilities to measure the sweat. In such exemplary aspects, it is understood that the skin does not interfere with the device's measurements.

In yet further aspects, the device comprises a sealing member 304 configured to encompass the sweat collector 308 and form a seal between the sweat collector and the subject's skin. In such aspects, the sealing member is configured to substantially seal the subject's skin around the sweat collector to minimize exposure of the sweat collector to the air and thus minimize a possible loss of the sweat from evaporation. In still further aspects, and as shown in FIG. 3B, the substrate 302 is configured to host the sealing member 304. It is understood that in certain aspects, the sealing member 304 can be replaced or reused if needed. In yet further aspects, and as also shown in FIG. 3B, at least a portion of the sealing member 304 can extend above the first portion of the sweat collector 307.

It is understood that the sealing member 304 can comprise any materials that can provide a substantial seal with the subject's skin. In yet further aspects, it is understood that the sealing member is chosen from any materials that do not negatively affect the subject's skin. In certain aspects, the sealing member can comprise an elastomer seal, a ridge knife-edge seal, an adhesive seal, or any combination thereof. In yet other exemplary aspects, the sealing member is a hydrophobic material. In yet other aspects, the sealing member comprises a material that is chemically inert with respect to sweat or any other materials that can be applied to the skin, such as for example, sunscreen, lotions, sanitizers, and the like.

In still further exemplary and unlimiting aspects, and as it can be seen in FIGS. 3A and 3D, at least a portion of the sealing member, can define a substantially circular sweat collection area. It is understood, however, that the seal can have any shape that would fit a specific application can comprise a substantially circular, rectangular, triangular, trapezoid, or any other irregular shape. In such aspects, at least a portion of the sealing member can also define a substantially rectangular, triangular, trapezoid, or any other irregular shape sweat collection area.

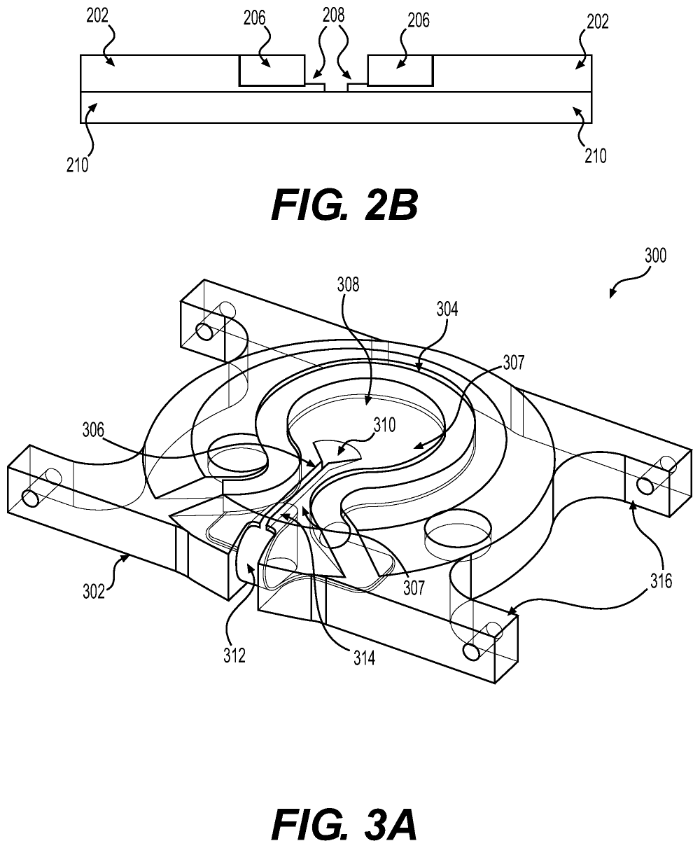

A top view of one of the exemplary devices is also shown in FIG. 2A (left), in which the sealing member 202 forms a substantially circular a sweat collecting area

Still, in further aspects, the device comprises an open channel 306 (FIGS. 3A and 3B). It is understood that the channel is an open channel because at least a portion of the channel is open to the ambient atmosphere. In still other exemplary aspects, the open channel allows the channel to stay substantially clean and unclogged. In yet further aspects, the open channel can have a width and a height. In yet further aspects, the open channel 306 can be formed within the substrate 302. While in other aspects, the open channel can comprise a rigid material. It is understood that the rigid material of the open channel can be the same or different from the rigid material of the sweat collector and/or the substrate.

In yet other aspects, the open channel can be formed in a material that is different from the substrate. For example, and without limitation, the open channel can be formed by the sealing member itself. In such an exemplary and unlimiting aspect, at least a portion of the sealing member can have a slit having a width that would define the width of the open channel. In yet other aspects, if the open channel is defined by the slit in the sealing member, the height of the open channel can be defined by a portion of the sealing member extending above the sweat collector and the subject's skin. In still further aspects, the sealing member comprises any material that allows comfortable use by the subject.

In still further aspects, the height of the open channel can be from about 100 μm to about 300 μm, including exemplary values of about 110 μm, about 120 μm, about 130 μm, about 140 μm, about 150 μm, about 160 μm, about 170 μm, about 180 μm, about 190 μm, about 200 μm, about 210 μm, about 220 μm, about 240 μm, about 250 μm, about 260 μm, about 270 μm, about 280 μm, and about 290 μm. It is further understood that the open channel can have any height value between any two foregoing values.

In still further aspects, the width of the open channel can be anywhere between about 100 μm to about 700 μm, including the exemplary value of about 150 μm, about 200 μm, about 250 μm, about 300 μm, about 350 μm, about 400 μm, about 450 μm, about 500 μm, about 550 μm, about 600 μm, and about 650 μm. It is further understood that the open channel can have any width value between any two foregoing values.

It is understood that the open channel width and height can be adjusted to suit a particular application.

In still further aspects, the open channel can have an aspect ratio of the height to the width no greater than about 10, no greater than 9, no greater than 8, no greater than 7, no greater than 6, no greater than 5, no greater than 4, no greater than 3, no greater than 2, or no greater than 1. It is understood that these exemplary aspect ratios of the open channel height to the open channel width ensure that the open channel is substantially unclogged during the life cycle of the device.

In still further aspects, the open channel can have a cross-section area of about 0.01 mm² to about 0.1 mm², including exemplary values of about 0.02 mm², about 0.03 mm², about 0.04 mm², about 0.05 mm², about 0.06 mm², about 0.07 mm², about 0.08 mm², and about 0.09 mm². It is further understood that the open channel can have any cross-section area between any two foregoing values.

It is understood that the collection area of the device can be determined based on device usability. For example, the lag time for initial reading from low sweat rates and response to changes in salt concentration need to be taken into consideration to determine the sweat collection area. In yet other aspects, a flow rate within the open channel needs to be considered to determine the specific size of the sweat collection area. In still further aspects, the sweat collection area can be anywhere between about 0.1 to about 10 cm², including exemplary values of about 0.5 cm², about 1 cm², about 2 cm², about 3 cm², about 4 cm², about 5 cm², about 6 cm², about 7 cm², about 8 cm², and about 9 cm². It is further understood that the open channel can have any sweat collection area between any two foregoing values.

In yet further aspects, the cross-section area of the open channel is about 100 to about 10,000 times smaller than the sweat collection area. In yet further aspects, the cross-section area of the open channel is about 100, about 250, about 500, about 750, about 1,000, about 1,500, about 2,000, about 2,500, about 3,000, about 3,500, about 4,000, about 4,500, about 5,000, about 5,500, about 6,000, about 7,500, about 8,000, about 8,500, about 9,000, about 9,500, or about 10,000 times smaller than the sweat collection area.

In still further aspects, the open channel is configured to continuously receive and transfer through an aliquot of a collected sweat from the sweat collector 308. It is understood that the aliquot of the collected sweat can comprise any amount effective to provide a desired sensory response. In still further aspects, the open channel of the disclosed device is configured to transfer through the aliquot of sweat at a flow rate to prevent back-diffusion.

Also disclosed herein are aspects where the device can comprise two or more channels. In some exemplary aspects, if at least two channels are present, they can be interconnected. While in other aspects, the at least two channels are not interconnected. In still further aspects, if more than two channels are present, some of the channels can be interconnected while others are not. In certain aspects, the two or more channels can be formed within the substrate. In yet other aspects, the two or more channels can be formed within a rigid material that is different from the substrate. In still further aspects, if two or more channels are present, one of the channels, for example, can be formed within the sealing member, while another channel can be formed within the substrate or any other rigid material. In yet other aspects, when two or more channels are present, each channel is an open channel. Yet, in other aspects, when two or more channels are present, at least one channel is an open channel, while other channels can be either open or closed or a combination thereof.

In still further exemplary and unlimiting aspects, the sweat collector can also comprise additional portions configured to help deliver the aliquot of sweat to the open channel. In certain exemplary and unlimiting aspects, the sweat collector can comprise a decreasing tapered ramp 310 between a portion of the first portion 307 and the open channel 306. In such aspects, the decreasing tapered ramp can help to guide the sweat from the collection area towards the open channel. In still further aspects, at least a portion of the first portion can comprise a roughness that is substantially similar to a typical roughness of the skin, from about 20 μm to about 40 μm, including exemplary values of about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, about 35 μm, about 36 μm, about 37 μm, about 38 μm, and about 39 μm. In such exemplary aspects, the roughness that can be present at at least a portion of the first portion can promote the flow of the collected sweat to the channel.

In yet other aspects, the minimal roughness can be defined by limitations of the manufacturing processes used to form the disclosed herein device. It is further understood that the surface roughness of the skin can also determine the dead volume that needs to be filled with sweat before it starts to flow through the channel, and a rough surface to the collection area would add to this dead volume. The surface roughness of the skin can be increased in subjects with coarse body hair on the area being measured.

In still further aspects, the open channel can comprise an outlet. In such exemplary aspects, the outlet of the open channel can be in communication with an ambient atmosphere. While in other aspects, the outlet of the open channel can also be in communication with an additional member, wherein the additional member is positioned outside of the device and is configured to collect the sweat exiting the open channel. It is understood that this additional member can be any member that is configured to collect the sweat. In some aspects, the additional member can comprise a container, a channel, a detector, or any combination thereof. In certain aspects, the sweat collected in this additional member can be further analyzed. In certain aspects, the additional member can be removably attached to the device. While in other aspects, the additional member can be permanently attached to the device.

In still further aspects, the device having the disclosed configuration is substantially clogging-free. In such aspects, even when the open channel is full of sweat and subsequently left to dry, the disclosed configuration and the disclosed aspect ratio ensure that the open channel is not blocked by dried sweat, primarily salt crystals. Any dried-on salt can dissolve quickly in the first sweat received and be rapidly flushed away, thus not affecting the subsequent measurement of the sweat. In yet further aspects, the disclosed open geometry allows easy cleaning of the open channel due to blockage by non-dissolvable materials (skin residue).

In yet further aspects, the aliquot is delivered to a sensing area 204, 210 (FIG. 2 A-left and right; FIG. 2B) defined by at least a portion of the flat portion of the sweat collector 206 and the sealing member 202 and comprises an exemplary sensor 208. Another exemplary sensing area 314 is also shown in FIGS. 3A and 3C.

In still further aspects, the disclosed herein device can comprise a sensor, for example, a sensor 208 shown in FIGS. 2A and 2B can be in fluid communication with the collected sweat (not shown) and is configured to detect at least one property of the collected sweat. In such exemplary aspects, the sensor 208 can be positioned at a distance from the first portion 206 such that the sensor has substantially no contact with the subject's skin. It is understood that the sensor is positioned within the sensing area such that there is substantially no contact between the sensor and the skin.

In still further aspects, the device can comprise two or more sensors. In such exemplary aspects, the two or more sensors can be the same or different. In yet further aspects, the two or more sensors can detect the same property of the collected sweat or a different property. In yet further aspects, even if the two or more sensors are different, these two sensors still can detect the same property.

It is further understood that the sensor can be positioned anywhere as long as it is in fluid communication with the sweat and has substantially no contact with the subject's skin. In certain aspects, the sensor 208 can be positioned with the open channel (FIGS. 2A-B). Yet, in other aspects, the sensor can be positioned adjacent to an inlet of the open channel. Yet, in other aspects, the sensor can be positioned adjacent to an outlet of the open channel. In yet other aspects, the sensor can be positioned on a sidewall of the open channel. In yet further unlimiting aspects, the sensor can be positioned on a surface of the open channel, wherein the distance from the first portion is substantially equal to the height.

A photograph of an exemplary prototype is shown in FIG. 3D.

In still further aspects, any properties of the sweat can be measured. In some aspects, the at least one property of the sweat can be selected from impedance, conductivity, refraction, temperature, or a combination thereof. In yet further embodiments, the at least one property that can be measured is impedance. In yet further aspects, when two or more sensors are present, the sweat collector can be configured to behave as a part of a sensor that is different from the impedance sensor. For example, and without limitations, the sweat collector can be formed on a glass or any other optically transparent material and can operate as an optical heart rate sensor.

Sweat generally comprises an electrolyte solution comprising alkali and alkali earth metal cations and their conjugated anions, such as, for example, chlorides. It is known that impedance measurements can be useful in evaluating the conductivity and concentration of the electrolytes as well as evaluating the bulk transport properties of the materials. In certain aspects, the impedance sensor can be configured to detect an amount of sodium ion and/or an amount of chloride ion in the collected sweat. In yet other aspects, the impedance sensor can be configured to detect a change in an amount of sodium ion and/or an amount of chloride ion in the collected sweat. In still further aspects, the impedance sensor can measure a change in the sweat rate of the body of the subject or the rate at which the subject is excreting sweat or sodium from his body or cells.

The impedance sensor used herein can comprise two electrodes, each having an electrode length and positioned opposing each other at an electrode gap.

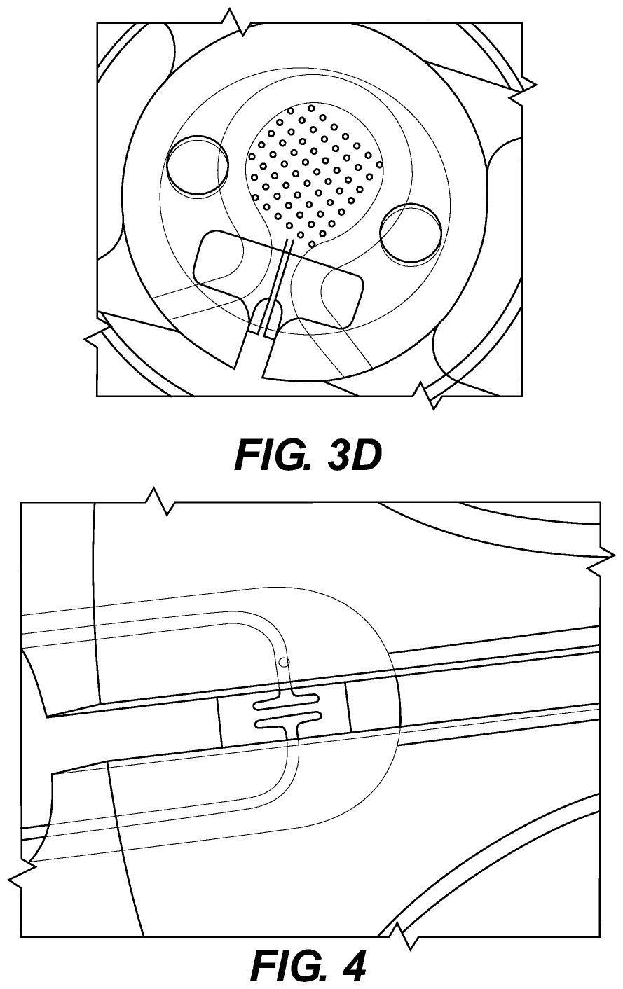

In still further aspects, the two electrodes can have any shape that allows effective measurement of impedance. In certain aspects, the electrodes can have a T-shape. A photograph of an exemplary impedance sensor comprising T-shaped electrodes is shown in FIG. 4. It is understood that current flows from the entire exposed area of one electrode to the other rather than just from the tips of the electrodes. In certain exemplary embodiments, the use of T-shaped electrodes ensures that most of the current takes the shortest path between electrodes, mostly independent of the overall exposed area.

In still further aspects, the electrodes can have an inert conductive coating. In certain aspects, the conductive coating can comprise gold. In yet other aspects, the conductive coating can comprise any precious metals, for example, platinum, palladium, rhodium, osmium, or alloys thereof, titanium, indium tin oxide, zin oxide, chromium nitride, conductive polymers, or carbon-based materials such as carbon, black carbon, graphite, graphene, and the like. In still further aspects, it is understood that any conductive and non-corroding coating can be used. In still further exemplary aspects, a coated substrate such as a conductively coated glass can be used as electrodes, or transparent conductive oxides can be used as electrodes.

In certain aspects, the electrode gap is from about 50 μm to about 500 μm, including exemplary values of about 100 μm, about 150 μm, about 200 μm, about 250 μm, about 300 μm, about 350 μm, about 400 μm, and about 450 μm.

In yet further aspects, the electrodes are disposed at a distance from the first portion at about 0.1 to about 1 mm, including exemplary values of about 0.2 mm, about 0.3 mm, about 0.4 mm, about 0.5 mm, about 0.6 mm, about 0.7 mm, about 0.8 mm, and about 0.9 mm.

In yet further aspects, the distance from the first portion is greater than the electrode gap.

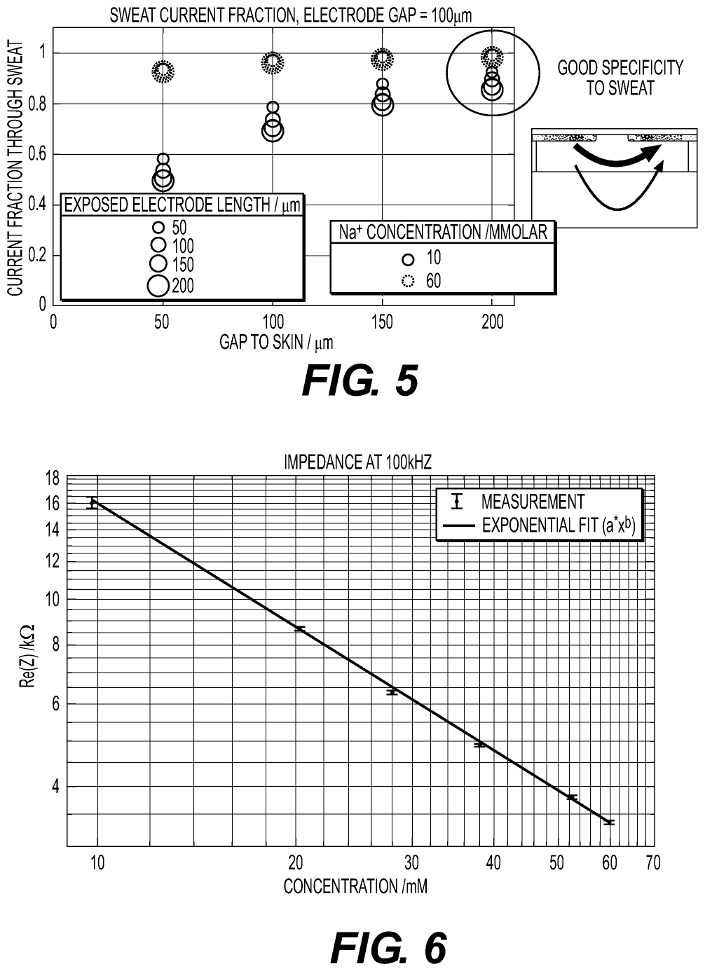

It is further understood that the distance from the first portion, as well as the gap between the electrodes, can be adjusted to ensure that no interference from the skin is measured. FIG. 5 shows a simulation of robustness to interference from the skin for different electrode spacings and gaps to the skin at two different Na⁺ concentrations of 10 mM and 60 mM. It can be seen, for example, that at a height to the skin (or alternatively the distance from the first portion of the device) of about 200 μm, the sensor has comparatively low interference from the skin, even for the most dilute (resistive) sweat.

In yet further aspects, the electrode length is from about 0.1 to about 10 mm, including exemplary values of about 0.5 mm, about 1 mm, about 1.5 mm, about 2 mm, about 2.5 mm, about 3 mm, about 3.5 mm, about 4 mm, about 4.5 mm, about 5 mm, about 5.5 mm, about 6 mm, about 6.5 mm, about 7 mm, about 7.5 mm, about 8 mm, about 8.5 mm, about 9 mm, and about 9.5 mm. In yet further aspects, the electrode length can be tuned to adjust the measured impedance to a range easily measured by low-cost integrated electronics without affecting the operational frequency.

It is further understood that the gap between the two electrodes can be tuned to adjust the required AC frequency range to be easily measured by low-cost integrated electronics. Without wishing to be bound by any theory, it is understood that the larger the gap, the lower the minimum frequency can be used for the measurements without encountering parasitic capacitances due to the double layer effect. It is further understood that the smaller the gap, the closer the sensor can be brought to the skin without sacrificing specificity to sweat.

In certain aspects, the impedance sensor can operate at a frequency equal to or less than about 1000 kHz, less than about 900 kHz, less than about 800 kHz, less than about 700 kHz, less than about 600 kHz, less than about 500 kHz, less than about 400 kHz, less than about 300 kHz, less than about 200 kHz, less than about 100 kHz, less than about 90 kHz, less than about 80 kHz, less than about 70 kHz, less than about 60 kHz, less than about 50 kHz, less than about 40 kHz, less than about 30 kHz, less than about 20 kHz, or less than about 10 kHz.

In yet further aspects, the impedance sensor operates at a frequency from about 10 kHz to about 100 kHz, including exemplary values of about 20 kHz, about 30 kHz, about 40 kHz, about 50 kHz, about 60 kHz, about 70 kHz, about 80 kHz, and about 90 kHz. In yet further aspects, the impedance sensor can operate at any of the frequencies disclosed above or at any frequency between any two foregoing values. In still further aspects, the impedance sensor can operate at two or more frequencies having any values as disclosed above.

In still further aspects, the impedance sensor disclosed herein can measure the impedance of the sweat at a voltage of less than about 1 Volt, less than about 0.9 Volt, less than about 0.8 Volt, less than about 0.7 Volt, less than about 0.6 Volt, less than about 0.5 Volt, less than about 0.4 Volt, less than about 0.3 Volt, less than about 0.2 Volt, or less than about 0.1 Volt. It is further understood that at these operating conditions, substantially no bubbles due to electrolysis of the sweat are formed within the aliquot of the sweat.

In still further aspects, the impedance sensor is configured to measure the resistivity of the sweat, which can be greater than about 1 kOhm, greater than about 1.5 kOhm, greater than about 2 kOhm, greater than about 2.5 kOhm, greater than about 3 kOhm, greater than about 3.5 kOhm, greater than about 4 kOhm, greater than about 4.5 kOhm, greater than about 5 kOhm, greater than about 5.5 kOhm, greater than about 6 kOhm, greater than about 6.5 kOhm, greater than about 7 kOhm, greater than about 7.5 kOhm, greater than about 8 kOhm, greater than about 8.5 kOhm, greater than about 9 kOhm, greater than about 9.5 kOhm, or greater than about 10 kOhm. It is understood that the higher resistivity is measured for sweat having smaller concentrations of the electrolyte.

In still further aspects, the impedance sensor, as disclosed herein, can measure the impedance of the sweat at any appropriate rate. For example, in some aspects, the sensor is configured to measure an impedance of the sweat at a rate comparable to a filling time of the open channel. In yet other aspects, the impedance measurement may be performed at a rate determined by the rate of change of previous impedance measurements.

In still further aspects, the device can comprise at least one additional sensor that is different from the impedance sensor. Such an additional sensor can be any sensor configured to measure the biological response of the subject. For example, the additional sensor can be a heart rate sensor or an oxygen saturation sensor.

In such exemplary aspects, the impedance sensor can be configured to measure an impedance of the sweat at a rate determined by a rate of measurement of the at least one additional sensor. For example, and without limitation, the device can comprise a heart rate sensor configured to measure periods of elevated heart rate. In such aspects, the heart rate elevation measurements over the resting period can be used to determine the likely sweating rate and thereby to determine the needed sampling rate. For example, and without limitation, when during walking, the heart rate elevation is low, a low update rate could be used for the sweat sensor. Yet, for example, when during running, the heart rate elevation is higher, a faster rate of measuring the sweat concentration could be used.

In yet further aspects, the device disclosed herein is configured to detect electrode wetting and contamination based on the impedance measurements. More specifically, the contamination and/or level of wetting can be determined by measuring impedance at at least two different frequencies and comparing real and imaginary components of the measured impedance to determine correlation with a trend as shown below.

The device disclosed herein is configured to operate in continuous or discrete mode depending on the desired application. In yet further aspects, the device is configured to provide time-resolved measurements.

In still further aspects, the device, as disclosed herein, has a low dead volume. In yet other aspects, the device disclosed herein is substantially robust to water evaporation during exercise due to substantially fast flow rates. In still further aspects, the device disclosed herein is substantially robust to electrical interference from the skin.

In yet further aspects, the device is configured to self-clean. The self-cleaning of the device can be accomplished by any known in the art methods. For example, and without limitations, the self-cleaning can be accomplished by applying voltage pulses, for example, to cause electrolysis on the electrode surfaces, providing both a chemical (acid, chlorine production) and physical cleaning (due to bubble formation). It is understood that the voltage needed for the self-cleaning operation can be the same or different from the voltage used for the impedance measurements. In certain aspects, the voltage needed for the self-cleaning operation can be greater than about 0.1 V, greater than about 0.2 V, greater than about 0.3 V, greater than about 0.4 V, greater than about 0.5 V, greater than about 0.6 V, greater than about 0.7 V, greater than about 0.8 V, greater than about 0.9 V, greater than about 1 V, greater than about 1.2 V, greater than about 1.5 V, greater than about 2 V, or greater than about 5 V.

In still further aspects, the device is configured to detect natural sweating, medically induced sweating, or a combination thereof. In certain aspects, the device disclosed herein can indicate the subject's level of hydration or whether the subject has any medical conditions affecting their sweat.

In yet further aspects, the sensing area can also comprise a control unit configured to analyze and process signals obtained from the sensor. In such aspects, the sensor is in at least electrical communication with the control unit. It is understood that the control unit can be permanently or detachably attached to the device. In yet further aspects, the sensor can be in communication with a user interface.

In certain aspects, the control unit of the disclosed device can further comprise computing and data storage capability sufficient to operate the device. In yet further aspects, the control unit incorporates the ability to conduct communication among system components, to perform data aggregation, and to execute algorithms capable of generating notification messages that can be shown on the user interface, for example. In certain aspects, notification messages can be visual. While in other aspects, the notification messages can be auditory.

In still further aspects, the device can have varying degrees of onboard computing capability (i.e., processing and data storage capacity). For example, all computing resources could be located onboard the device, or some computing resources could be located on a disposable portion of the device and additional processing capability located on a reusable portion of the device. Alternatively, the device may rely on portable, fixed, or cloud-based computing resources.

The device is further configured to aggregate the measured data and compare various measurements over a period of time. It is further understood that since the sweat comprises the subject's physiological data, the device can be configured to de-identify the data from the subject or it could remain associated with the subject.

In still further aspects, the devices control unit is configured to correlate sweat measurements with outside information, such as the time, date, air temperature, humidity, activity performed by the individual, motion level, fitness level, mental and physical performance during the data collection, body orientation, the proximity to significant health events or stressors, age, sex, medications, drug sensitivity, medical condition, health history, or other relevant information.

In certain aspects, the device is configured to be worn on the subject's skin (as shown, for example, in FIG. 3A, where the substrate of the device comprises connections 316 to a wrist band) and measure sweat properties continuously or discretely. Yet, in other aspects, the device can be integrated within the handheld device, and the sweat properties can be measured per the subject's request.

In still further aspects, the data collected by the device can be transferred to any other medium, such as a secure website, a CD, a flash drive, etc. In yet further aspects, the sweat data monitored by the user can include real-time data, trend data, or may also include aggregated sweat data drawn from the system database and correlated to the subject's sex or fitness level, weather condition, activity, combined analyte profile, or other relevant metric. Trend data, such as a target subject's hydration level over time, could be used to predict future performance or the likelihood of an impending physiological event. Sweat data can also be used to identify wearers that are in need of additional monitoring or instruction, such as the need to drink additional water or to adhere to a drug regimen.

In yet further aspects, the device described herein is not limited to measurement of the sweat and can be adapted for measurement of any other appropriate fluid, whether it is a biological fluid or not.

### Methods

Also disclosed herein are methods of making the disclosed devices. In certain aspects, the methods comprise a) forming a sweat collector on a rigid material; b) positioning a sealing member such that the sealing member is configured to encompass the sweat collector; c) forming an open channel, wherein the open channel has a width and a height, wherein the open channel has an aspect ratio of the height to the width no greater than about 10; wherein the open channel is configured to continuously receive and transfer through an aliquot of a collected sweat from the sweat collector; and d) positioning a sensor such that the sensor is positioned at a distance from the first portion such that the sensor has substantially no contact with the subject's skin and such that the sensor is in fluid communication with the collected sweat and is configured to detect at least one property of the collected sweat.

It is understood that a rigid material can be any material known in the art and disclosed herein. In yet further aspects, the sweat collector and/or open channel formed on the rigid material can be formed by any methods known in the art. For example, and without limitations, the sweat collector and the open channel can be formed by etching a rigid material, laser cutting, or using 3D printing. It is further understood that the etching can be any etching known in the art depending on the specific rigid material, for example, it can be solution etching, plasma-assisted etching, photoetching, and the like. In yet other aspects, the sweat collector, as well as the open channel, can be formed by injection molding, casting, embossing, or 3D printing.

In yet further aspects, the sealing member can be preformed prior to positioning it such that it seals the sweat collector against the subject's skin. It is understood that it can be preformed to any shape as disclosed above and as desired by the specific application. In yet further aspects, the sealing member can comprise any materials as disclosed above that provide for a substantial seal and are comfortable to the subject, and do not cause any negative effects to the subject's skin.

In certain aspects and as disclosed above, the open channel can be formed by other methods that are different from forming the open channel on the rigid material. For example, and without limitations, the sealing member can comprise a slit having a width, as disclosed above. In such aspects, the slit can define the open channel.

In yet further aspects, and as disclosed above, the sensor can be an impedance sensor. In such aspects, the sensor can be preformed, and then the preformed sensor can be positioned in the desired location on the device. Any of the possible sensor locations described above can be applicable.

In certain aspects, the sensor is preformed by forming two opposite electrode tracks on printed circuit board materials (PCB). In certain aspects, the PCB can be a rigid PCB or a flexible PCB (or Flexifoil as illustrated, for example, in FIGS. 3D and 4). It is understood that any standard PCB processes can be utilized to form these electrode tracks. In still further aspects, the standard PCB processes allow formation of the features having a size equal to or less than about 50 μm. It is understood, however, that PCB processes are exemplary only, and any known in the art methods of forming micro-sized electrodes can be utilized. For example, and without limitations, any known processes used in the semiconductor industry, electrochemical industry, or metallurgical industry, or any other industry can be utilized. For example, the electrodes can be formed by etching, plating, metal sputtering, laser ablation, damascene processing, and the like. For example, and without limitations during a damascene process, electrodes are formed by forming grooves in an insulating process, followed by metal plating, and surface polishing

In other aspects, the electrode tracks are coated with any known inert conductive material. For example, and without limitations, the inert conductive material can comprise any precious metals, for example, gold, platinum, palladium rhodium, osmium, or alloys thereof, titanium, indium tin oxide, zin oxide, chromium nitride, conductive polymers, or carbon-based materials such as carbon, black carbon, graphite, graphene, and the like. In still further aspects, it is understood that any conductive and non-corroding coating can be used. In still further exemplary aspects, a coated substrate such as a conductively coated glass can be used as electrodes, or transparent conductive oxides can be used as electrodes

In still further aspects, the formed electrodes can have a shape known in the art. In certain aspects, the methods disclosed herein comprise forming T-shape electrodes. In certain aspects, the electrodes of the current disclosure are formed such that most of the current takes the shortest path between electrodes and, therefore, is mostly independent of the overall exposed area. It is understood that the shape of the electrodes, as disclosed herein, can compensate for possible inaccuracy of the PCB processing methods or alignment of the sensing area with the open channel. In some aspects, the electrode's shape is designed to mitigate the impact of inaccuracies in PCB processing methods.

In yet further aspects, the methods disclosed herein allow substantial control of an electrode gap and length. For example, and as disclosed above, the methods disclosed herein allow formation of the electrodes having the electrode gap from about 50 μm to about 500 μm, including exemplary values of about 100 μm, about 150 μm, about 200 μm, about 250 μm, about 300 μm, about 350 μm, about 400 μm, and about 450 μm.

While in still further aspects, and as disclosed above, the methods disclosed herein allow the formation of the electrodes having the electrode length from about 0.1 to about 10 mm, including exemplary values of about 0.5 mm, about 1 mm, about 1.5 mm, about 2 mm, about 2.5 mm, about 3 mm, about 3.5 mm, about 4 mm, about 4.5 mm, about 5 mm, about 5.5 mm, about 6 mm, about 6.5 mm, about 7 mm, about 7.5 mm, about 8 mm, about 8.5 mm, about 9 mm, and about 9.5 mm.

In still further aspects, the methods disclosed herein allow using the disclosed herein device with off-the-shelf, low-cost electronics.

Also disclosed herein are methods of measuring the sweat. In such exemplary aspects, any of the disclosed above devices can be positioned on a subject's skin such that the sealing member seals the sweat collector against the subject's skin. The sweat is then collected within a sweat collection area, and at least one property is measured. It is understood that the sensors disclosed herein reconfigured to provide one or more output signals, wherein these output signals can be correlated with the at least one property of the sweat.

In still further aspects, the step of measuring can also include a step of correcting the one or more output signals for an artifact signal due to lack of electrode wetness and/or contamination. In yet further aspects, the step of measuring can be continuous or discrete.

In still further aspects, the one or more output signals can be collected and aggregated as described above. Yet, in other aspects, the one or more output signals can be transmitted to a user interface to produce an information display indicative of the hydration status of the subject.

### EXAMPLES

The following examples are put forth so as to provide those of ordinary skill in the art with a complete disclosure and description of how the compounds, compositions, articles, devices, and/or methods claimed herein are made and evaluated and are intended to be purely exemplary and are not intended to limit the disclosure. Efforts have been made to ensure accuracy with respect to numbers (e.g., amounts, temperature, etc.), but some errors and deviations should be accounted for.

### Example 1

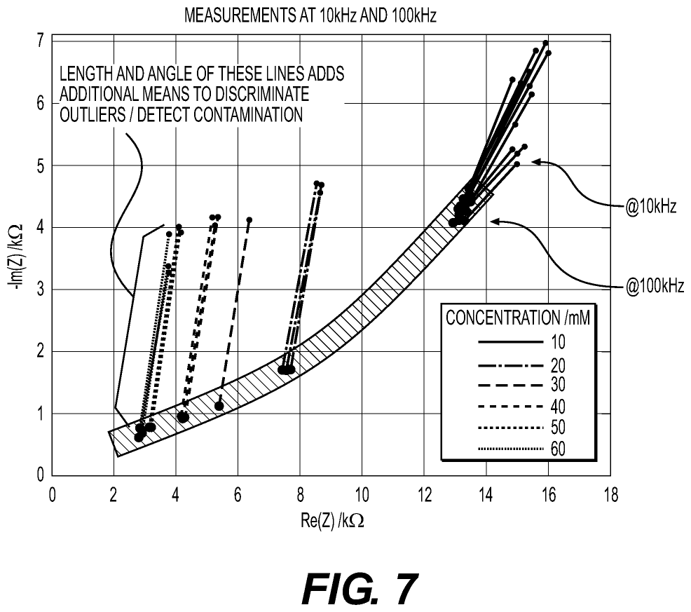

A correlation between the impedance measurements and the various concentrations was evaluated. FIG. 6 shows exemplary impedance measurements over a range of physiologically realistic sweat concentrations at 100 kHz. Each point is in triplicate, with error bars shown, showing the high accuracy of the measurement. It was found that this accuracy is maintained after drying the sweat residue within the open channel and remeasuring a newly supplied sweat.

### Example 2

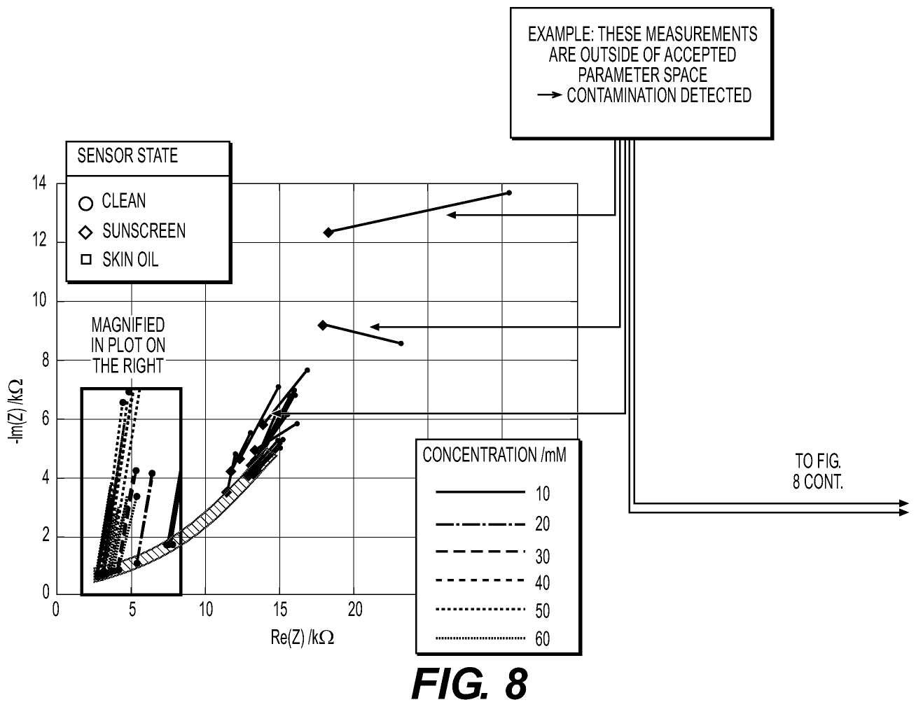

To evaluate the ability of the sensors to determine the level of electrodes' wetting and/or contamination, the impedance of various saline solutions mimicking the sweat was measured, and the results are shown in FIG. 7. Measurements were made at both 10 kHz and 100 kHz (small and large circles, respectively). The shaded green area represents the region that impedance measurements at 100 kHz are expected to fall in. The additional measurement at 10 kHz has served as an assurance. Without wishing to be bound by any theory, it was hypothesized that even if a contaminated electrode still provides a measurement that happens to fall within the shaded region, it would be unlikely that both the measurement at 100 kHz as well as the respective 10 kHz fall into the expected range unless there are no significant contaminants. In order to determine which measurement is a result of contaminated electrodes, the impedance can be measured at two different frequencies (e.g., 10 kHz and 100 kHz), and results are plotted on a Nyquist plot of −Im(Z) vs. Re(Z). A straight line between these two impedance measurements should be then considered. Such a line is defined by a specific length and a specific angle. The contamination is determined if the length of the specific measurement is outside of +/−4% of the expected value and/or the angle of the specific measurement is outside of +/−3 degrees of the expected value, wherein the expected value is determined based on calibration process, or modeling of the electrodes' geometry. Due to variations in the manufacturing process, in certain aspects, it can be sufficient to calibrate the design once, while in other aspects, each production batch can be calibrated once, or each device could be individually calibrated. The level of calibration can depend on both the variability of the process and the accuracy desired in the application.

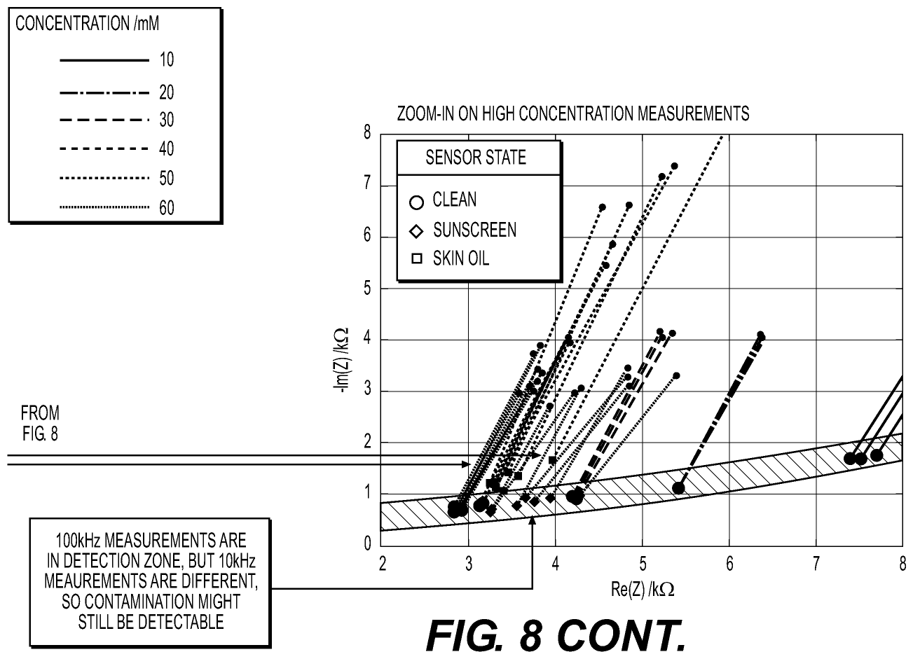

In still further aspect, the bounds for determining the deviation from the expected values can be determined by repeated experiments on contaminated and uncontaminated electrodes, as for example, it is shown in FIG. 8.

For example, a 60 mM saline sample contaminated with sunscreen was measured in one instance to have an impedance at 100 khz of Z=4.2+i1 kOhm, which is very similar to an uncontaminated sample of 40 mM saline (Z=4.2+i0.95 kOhm), but the length (L) and angle (Phi) described above were found to be outside of the specified regions of interest: measured L(contaminated)=2.6 kOhm, Phi(contaminated)=65 degrees in comparison to the expected L(clean)=3.4+/−0.14 kOhm, Phi(Clean)=72.2+/−3 degrees.

Contamination results are shown in FIG. 8. Severe levels of skin oil and sunscreen were applied to the detector to evaluate the interference. At high NaCl concentration, severe sunscreen contamination could lead to an underestimation of sweat concentration by up to 30%. While the contaminated electrodes can provide readings in the expected range at 100 kHz, the difference between 100 kHz and 10 kHz measurements differs for contaminated electrodes, allowing contamination to be detected (plot on the right, diamond and square symbols highlighted with arrows).

The claims are not intended to include, and should not be interpreted to include, means-plus- or step-plus-function limitations, unless such a limitation is explicitly recited in a given claim using the phrase(s) “means for” or “step for,” respectively.

In view of the described processes and compositions, hereinbelow are described certain more particularly described aspects of the inventions. These particularly recited aspects should not, however, be interpreted to have any limiting effect on any different claims containing different or more general teachings described herein, or that the “particular” aspects are somehow limited in some way other than the inherent meanings of the language and formulas literally used therein.

### Aspects:

Aspect 1: A device comprising: a) a sweat collector having a shape, wherein at least a portion of the sweat collector comprises a first portion configured to face and to conform to a subject's skin; b) a sealing member configured to encompass the sweat collector and form a seal between the sweat collector and the subject's skin; c) an open channel having a width and a height, wherein the open channel has an aspect ratio of the height to the width no greater than about 10; wherein the open channel is configured to continuously receive and transfer through an aliquot of a collected sweat from the sweat collector; and d) a sensor that is in fluid communication with the collected sweat and is configured to detect at least one property of the collected sweat, wherein the sensor is positioned at a distance from the first portion such that the sensor has substantially no contact with the subject's skin.

Aspect 2: The device of Aspect 1, wherein at least a portion of the sealing member extends above the first portion of the sweat collector.

Aspect 3: The device of Aspect 1 or 2, wherein the sealing member comprises an elastomeric seal, a ridge knife-edge seal, an adhesive seal, or any combination thereof.

Aspect 4: The device of any one of Aspects 1-3, wherein the sealing member comprises a hydrophobic material.

Aspect 5: The device of any one of Aspects 1-4, wherein at least a portion of the sealing member defines a substantially circular sweat collection area.

Aspect 6: The device of any one of Aspects 1-5, wherein the sweat collector comprises a decreasing tapered ramp between a portion of the first portion and the open channel.

Aspect 7: The device of any one of Aspects 1-6, wherein the sweat collector and the open channel comprise a rigid material.

Aspect 8: The device of any one of Aspects 1-5, wherein a portion of the seal has a slit having a thickness and wherein the slit defines the open channel.

Aspect 9: The device of any one of Aspects 1-8, wherein the open channel has a height of about 100 μm to about 300 μm.

Aspect 10: The device of any one of Aspects 1-9, wherein the open channel has a width of about 100 μm to about 700 μm.

Aspect 11: The device of any one of Aspects 1-10, wherein an outlet of the open channel is in communication with an ambient atmosphere.

Aspect 12: The device of any one of Aspects 1-10, wherein an outlet of the open channel is in communication with an additional member, wherein the additional member is positioned outside of the device and is configured to collect the sweat exiting the open channel.

Aspect 13: The device of any one of Aspects 1-12, wherein the open channel is configured to transfer through the aliquot of sweat at a flow rate to prevent back-diffusion.

Aspect 14: The device of any one of Aspects 1-13, wherein the open channel has a cross-sectional area of about 0.01 to about 0.1 mm².

Aspect 15: The device of any one of Aspects 1-14, wherein a sweat collection area is from about 0.1 to about 10 cm².

Aspect 16: The device of Aspect 15, wherein the cross-sectional area of the open channel is about 100 to about 10,000 times smaller than the sweat collection area.

Aspect 17: The device of any one of Aspects 1-16, wherein at least a portion of the first portion comprises a roughness configured to promote guiding of the collected sweat to the open channel.

Aspect 18: The device of any one of Aspects 1-17, wherein the device comprises two or more channels.

Aspect 19: The device of Aspect 18, wherein at least two channels are interconnected.

Aspect 20: The device of any one of Aspects 1-19, wherein the device comprises two or more sensors, wherein two or more sensors are the same or different.

Aspect 21: The device of any one of Aspects 1-20, wherein the sensor is positioned within the open channel.

Aspect 22: The device of any one of Aspects 1-21, wherein the sensor is positioned adjacent to an inlet of the open channel.

Aspect 23: The device of any one of Aspects 1-22, wherein the sensor is positioned adjacent to an outlet of the open channel.

Aspect 24: The device of any one of Aspects 1-23, wherein the sensor is positioned on a surface of the open channel, wherein the distance from the first portion is equal to the height.

Aspect 25: The device of any one of Aspects 1-24, wherein the at least one property of the sweat is selected from impedance, conductivity, refraction, temperature, or a combination thereof.

Aspect 26: The device of Aspect 25, wherein the at least one property of the sweat is impedance.

Aspect 27: The device of any one of Aspects 1-26, wherein the device is configured to detect natural sweating, a medically induced sweating, or a combination thereof.

Aspect 28: The device of any one of Aspects 1-27, wherein the device is configured to provide time-resolved measurements.

Aspect 29: The device of any one of Aspects 1-27, wherein the device is configured to self-clean.

Aspect 30: The device of any one of Aspects 26-29, wherein the sensor is an impedance sensor.

Aspect 31: The device of Aspect 30, wherein the impedance sensor is configured to measure at least one property at a voltage of less than about 1 Volt.

Aspect 32: The device of Aspect 30 or 31, wherein the impedance sensor is configured to detect an amount of sodium ion and/or an amount of chloride ion in the collected sweat.

Aspect 33: The device of any one of Aspects 30-32, wherein the impedance sensor comprises two electrodes, each having an electrode length and positioned opposing each other at an electrode gap.

Aspect 34: The device of Aspect 33, wherein the electrode gap is from about 50 to about 500 μm.

Aspect 35: The device of Aspect 33 or 34, wherein the electrode length is from about 0.1 to about 10 mm.

Aspect 36: The device of any one of Aspects 33-35, wherein the two electrodes have a T-shape.

Aspect 37: The device of any one of Aspects 33-36, wherein the two electrodes comprise an inert conductive coating.

Aspect 38: The device of any one of Aspects 1-37, wherein the distance from the first portion is from about 0.1 to about 1 mm.

Aspect 39: The device of any one of Aspects 34-38, wherein the distance from the first portion is greater than the electrode gap.

Aspect 40: The device of any one of Aspects 30-39, wherein the impedance sensor operates at a frequency equal to or less than 1,000 kHz.

Aspect 41: The device of Aspect 40, wherein the impedance sensor operates at a frequency from about 10 kHz to about 100 kHz.

Aspect 42: The device of any one of Aspects 30-41, wherein the impedance sensor operates at two or more frequencies.

Aspect 43: The device of any one of Aspects 30-42, wherein a measured resistivity is greater than about 1 kOhm.

Aspect 44: The device of any one of Aspects 30-43, wherein the device is configured to detect an electrode wetting and contamination.

Aspect 45: The device of any one of Aspects 30-44, wherein the impedance sensor is configured to measure an impedance of the sweat at a rate comparable to a filling time of the open channel.

Aspect 46: The device of any one of Aspects 30-45, wherein when the device comprises at least one additional sensor that is different from the impedance sensor, the impedance sensor is configured to measure an impedance of the sweat at a rate determined by a rate of measurement by the at least one additional sensor.

Aspect 47: The device of any one of Aspects 1-46, wherein the device is configured to operate in a continuous or discrete mode.

Aspect 48: The device of any one of Aspects 1-47, wherein the sensor is in communication with a control unit.

Aspect 49: The device of any one of Aspects 1-48, wherein the sensor is in communication with a user interface.

Aspect 50: The device of any one of Aspects 1-49, wherein the device is configured to be worn on the subject skin.

Aspect 51: The device of any one of Aspects 1-50, wherein the device comprises an information display configured to indicate a hydration status, wherein the hydration status of a subject is correlated to the at least one property of the sweat.

Aspect 52: A device comprising: a) a sweat collector having a shape, wherein at least a portion of the sweat collector comprises a first portion configured to face and to conform to a subject's skin; b) a sealing member configured to encompass the sweat collector and form a seal between the sweat collector and the subject's skin; c) an open channel having a width, and a height, wherein the open channel has an aspect ratio of the height to the width no greater than about 10; wherein the open channel is configured to continuously receive and transfer through an aliquot of a collected sweat from the sweat collector; and d) a sensor comprising two electrodes, each having an electrode length and positioned opposing each other at an electrode gap and are in fluid communication with the collected sweat and wherein the sensor is configured to detect an impedance of the collected sweat and wherein the sensor is positioned at a distance from the first portion such that the sensor has substantially no contact with the subject's skin.

Aspect 53: A method of measuring a sweat comprising: providing a device of any one of Aspects 1-52, wherein the device is positioned on a subject's skin such that the sealing member seals the sweat collector against the subject's skin; collecting the sweat within a sweat collection area; and measuring at least one property of the sweat by the sensor, wherein the at sensor provides one or more output signals correlated with the at least one property of the sweat.

Aspect 54: The method of Aspect 53, wherein the at least one property is an impedance of the sweat.

Aspect 55: The method of Aspect 54, wherein the sensor is an impedance sensor.

Aspect 56: The method of Aspect 55, wherein the step of measuring comprises measuring impedance at a voltage of less than about 1 Volt.

Aspect 57: The method of Aspect 55 or 56, wherein the step of measuring comprises measuring impedance at one or more frequencies in a range up to 1,000 kHz.

Aspect 58: The method of any one of Aspects 55-57, wherein the step of measuring comprises measuring impedance at two or more frequencies in a range from about 10 kHz to about 100 kHz.

Aspect 59: The method of any one of Aspects 53-58, wherein the step of measuring comprises correcting the output signal for an artifact signal due to lack of electrode wetness and/or contamination.

Aspect 60: The method of any one of Aspects 53-59, wherein the step of measuring is continuous or discrete.

Aspect 61: The method of any one of Aspects 53-60, transmitting the one or more output signals to a user interface to produce an information display indicative of the hydration status of the subject.

Aspect 62: A method of manufacturing the device of any one of Aspects 1-52, comprising: a) forming a sweat collector on a rigid material; b) positioning a sealing member such that the sealing member is configured to encompass the sweat collector; c) forming an open channel, wherein the open channel has a width and a height, wherein the open channel has an aspect ratio of the height to the width no greater than about 10; wherein the open channel is configured to continuously receive and transfer through an aliquot of a collected sweat from the sweat collector; and d) positioning a sensor such that the sensor is positioned at a distance from the first portion such that the sensor has substantially no contact with the subject's skin and such that the sensor is in fluid communication with the collected sweat and is configured to detect at least one property of the collected sweat.

Aspect 63: The method of Aspect 62, wherein the sealing member is preformed to a size.

Aspect 64: The method of Aspect 62 or 63, wherein at least a portion of the sealing member extends above the first portion of the sweat collector.

Aspect 65: The method of any one of Aspects 62-64, wherein the sealing member comprises an elastomeric seal, a ridge knife-edge seal, an adhesive seal, or any combination thereof.

Aspect 66: The method of any one of Aspects 61-65, wherein the sealing member comprises a hydrophobic material.

Aspect 67: The method of any one of Aspects 62-66, wherein the open channel is formed on the rigid material.

Aspect 68: The method of any one of Aspects 62-66, wherein the sealing member has a slit having a thickness and wherein the slit defines the open channel.

Aspect 69: The method of any one of Aspects 62-68, wherein the sensor is an impedance sensor.

Aspect 70: The method of any one of Aspects 62-69, wherein the sensor is preformed.

Aspect 71: The method of Aspect 69 or 70, wherein the impedance sensor is preformed by forming two opposite electrode tracks on a printed circuit material.

Aspect 72: The method of Aspect 71 further comprising forming two electrodes, each having an electrode length and positioned opposing each other at an electrode gap within the electrode tracks by depositing a conductive material.

Aspect 73: The method of Aspect 72, wherein the two electrodes have a T-shape form.

Aspect 74: The method of Aspect 72 or 73, wherein the electrode gap is from about 50 to about 500 μm.

Aspect 74: The method of any one of Aspects 71-74, wherein the electrode length is from about 0.1 to about 10 mm.

Aspect 75: The method of any one of Aspects 62-75, wherein the distance from the subject skin is from about 0.1 to about 1 mm.

Aspect 76: The method of any one of Aspects 71-76, wherein the distance from the first portion is greater than the electrode gap.

1. A device comprising:
a) a sweat collector, wherein at least a portion of the sweat collector comprises a first portion configured to face and conform to a subject's skin;
b) a sealing member configured to encompass the sweat collector and form a seal between the sweat collector and the subject's skin;
c) an open channel having a width and a height,
wherein the open channel has an aspect ratio of the height to the width no greater than about 10;
wherein the open channel is configured to continuously receive and transfer through an aliquot of a collected sweat from the sweat collector; and
d) a sensor that is in fluid communication with the collected sweat and is configured to detect at least one property of the collected sweat, wherein the sensor is positioned at a distance from the first portion such that the sensor has substantially no contact with the subject's skin.

2. The device of claim 1, wherein the sweat collector comprises a decreasing tapered ramp between a portion of the first portion and the open channel.

3. (canceled)

4. The device of claim 1, wherein a portion of the sealing member has a slit having a width and wherein the slit defines the open channel.

5. (canceled)

6. The device of claim 1, wherein the open channel is configured to transfer through the aliquot of the collected sweat at a flow rate to prevent back-diffusion.

7. The device of claim 1, wherein the open channel has a cross-sectional area of about 0.01 to about 0.1 mm².

8. The device of claim 1, wherein a sweat collection area is from about 0.1 to about 10 cm².

9. (canceled)

10. The device of claim 1, wherein the sensor is positioned:
a) within the open channel, adjacent to an inlet of the open channel, or adjacent to an outlet of the open channel;
b) or on a surface of the open channel, wherein the distance from the first portion is equal to the height.

11. The device of claim 1, wherein the at least one property of the sweat is selected from impedance, conductivity, refraction, temperature, or a combination thereof.

12. The device of claim 11, wherein the sensor is an impedance sensor configured to measure the at least one property at a voltage of less than about 1 Volt, and wherein the impedance sensor comprises two electrodes, each having an electrode length from about 0.1 to about 10 mm and positioned opposing each other at an electrode gap from about 50 to about 500 μm.

13. (canceled)

14. (canceled)

15. (canceled)

16. The device of claim 1, wherein the distance from the first portion is from about 0.1 to about 1 mm.

17. The device of claim 12, wherein the distance from the first portion is greater than the electrode gap.

18. The device of claim 17, wherein the impedance sensor operates at one or more frequencies from about 10 kHz to about 100 kHz.

19. (canceled)

20. (canceled)

21. The device of claim 12, wherein the device comprises at least one additional sensor that is different from the impedance sensor, and wherein the impedance sensor is configured to measure an impedance of the sweat at a rate determined by a rate of measurement of the at least one additional sensor.

22. The device of claim 1, wherein the device comprises an information display configured to indicate a hydration status, wherein the hydration status of a subject is correlated to the at least one property of the sweat.

23. A device comprising:
a) a sweat collector, wherein at least a portion of the sweat collector comprises a first portion configured to face and to conform to a subject's skin;
b) a sealing member configured to encompass the sweat collector and form a seal between the sweat collector and the subject's skin;
c) an open channel having a width and a height,
wherein the open channel has an aspect ratio of the height to the width no greater than about 10;
wherein the open channel is configured to continuously receive and transfer through an aliquot of a collected sweat from the sweat collector; and
d) a sensor comprising two electrodes, each defined by an electrode length and positioned opposing each other at an electrode gap and are in fluid communication with the collected sweat and wherein the sensor is configured to detect an impedance of the collected sweat and wherein the sensor is positioned at a distance from the first portion such that the sensor has substantially no contact with the subject's skin.

24. A method of measuring a sweat comprising:
providing a device of claim 1, wherein the device is positioned on a subject's skin such that the sealing member seals the sweat collector against the subject's skin;
collecting the sweat within a sweat collection area; and
measuring at least one property of the sweat by the sensor, wherein the at sensor provides one or more output signals correlated with the at least one property of the sweat.

25. (canceled)

26. The method of claim 24, wherein the step of measuring comprises measuring an impedance at a voltage of less than about 1 Volt and/or one or more frequencies in a range from about 10 kHz to about 100 kHz.

27. The method of claim 24, wherein the step of measuring comprises correcting the one or more output signals for an artifact signal due to lack of electrode wetness and/or contamination.

28. The method of claim 24, further comprising transmitting the one or more output signals to a user interface to produce an information display indicative of the hydration status of the subject.

29. A method of manufacturing a device, comprising:
a) forming a sweat collector on a rigid material;
b) positioning a sealing member such that the sealing member is configured to encompass the sweat collector;
c) forming an open channel, wherein the open channel has a width and a height,
wherein the open channel has an aspect ratio of the height to the width no greater than about 10;
wherein the open channel is configured to continuously receive and transfer through an aliquot of a collected sweat from the sweat collector; and
d) positioning a sensor such that the sensor is positioned at a distance from the first portion such that the sensor has substantially no contact with the subject's skin and such that the sensor is in fluid communication with the collected sweat and is configured to detect at least one property of the collected sweat.

30. (canceled)
